(19) United States
(12) Patent Application Publication
(10) Pub. No.: US 2018/0071240 A1
(43) Pub. Date: Mar. 15, 2018

(54) ONCE DAILY ORAL PHARMACEUTICAL COMPOSITION OF ISOTRETINOIN

(71) Applicant: SUN PHARMACEUTICAL INDUSTRIES LIMITED, Mumbai (IN)

(72) Inventors: Sumit MADAN, New Delhi (IN); Anuj Kumar FANDA, Ghaziabad (IN); Pankaj Prabhakar AMRUTKAR, Ahmednagar (IN); Rajesh RAO, Deoria (IN); Jitendra KAWADKAR, Betul (IN); Vasu Kumar KAKUMANU, Guntur (IN); Ravi KOCHHAR, Gurgaon (IN); Romi Barat SINGH, Varanasi (IN); Dileep JAMI, Srikakulam (IN); Lokesh GARG, Jind (IN); Sandipan ROY, Kolkata (IN)

(73) Assignee: SUN PHARMACEUTICAL INDUSTRIES LIMITED, Mumbai (IN)

(21) Appl. No.: 15/822,076

(22) Filed: Nov. 24, 2017

Related U.S. Application Data
(63) Continuation-in-part of application No. PCT/IB2016/053073, filed on May 25, 2016.

(30) Foreign Application Priority Data
May 25, 2015 (IN) 1470/DEL/2015

(51) Int. Cl. A61K 31/203; A61K 9/48; A61K 9/20; A61K 9/28

(52) U.S. Cl. CPC A61K 31/203; A61K 9/4891; A61K 9/2866; A61K 9/20; A61K 9/4825

(57) ABSTRACT

The present invention relates to a modified-release, once-daily, oral pharmaceutical composition comprising isotretinoin and a process for its preparation. In some embodiments, the disclosure is directed to a modified-release, oral pharmaceutical composition comprising isotretinoin or a pharmaceutically acceptable salt, ester or a derivative thereof; and a release modifying agent, wherein the composition is suitable for once daily administration. In some embodiments, the disclosure is directed to a modified-release, oral pharmaceutical composition comprising isotretinoin, wherein the composition comprises an immediate-release component; and a modified-release component comprising a release modifying agent, and wherein the composition is suitable for once daily administration.

### FIELD OF THE INVENTION

The present invention relates to a modified-release, once-daily, oral pharmaceutical composition comprising isotretinoin and a process for its preparation.

### BACKGROUND OF THE INVENTION

Isotretinoin is a retinoid, approved for the treatment of severe recalcitrant nodular acne. Chemically, isotretinoin is 13-cis-retinoic acid and is related to both retinoic acid and retinol (vitamin A). The present marketed dosage forms of isotretinoin require twice-daily administration based on the body weight of the patient.

There is a need to develop a more convenient method of administration which allows once-daily dosing of isotretinoin in order to have better patient compliance. To suffice this unmet need in the art, the present inventors have developed a modified-release, once-daily, oral pharmaceutical composition of isotretinoin.

### SUMMARY OF THE INVENTION

The present disclosure relates to a modified-release, oral pharmaceutical composition comprising isotretinoin or a pharmaceutically acceptable salt, ester or a derivative thereof; and a release modifying agent, wherein the composition is suitable for once daily administration.

The present disclosure relates to a modified-release, once-daily, oral pharmaceutical composition of isotretinoin with reduced food effects.

The present disclosure also provides a method for treating acne by administering the modified-release, oral pharmaceutical composition of isotretinoin once-daily.

### DETAILED DESCRIPTION OF THE INVENTION

As used herein, the word “a” or “plurality” before a noun represents one or more of the particular noun. For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

Unless otherwise defined, all technical and scientific terms used herein have the same meaning as commonly understood by one of ordinary skill in the art to which this disclosure belongs. Methods and materials are described herein for use in the present disclosure; other suitable methods and materials known in the art can also be used. The materials, methods, and examples are illustrative only and are not intended to be limiting. All publications, patent applications, patents, sequences, database entries, and other references mentioned herein are incorporated by reference in their entirety. In case of conflict, the present specification, including definitions, will control.

In one aspect, the present disclosure provides an oral pharmaceutical composition of isotretinoin suitable for once-daily administration.

In one embodiment of the above aspect, the oral pharmaceutical composition is a modified release composition.

In further embodiment, the said modified-release composition comprises a combination of an immediate-release component and a modified-release component.

Immediate-release component ensures release of isotretinoin not less than 25% in 4 hours. Immediate release component includes delayed release component. Immediate release component as used herein includes that composition comprises substantial amount of immediate release component. The phrase “substantial” means that the composition contains at least 5% of isotretinoin in the immediate release component.

In another embodiment of the above aspect, the modified-release component is an extended-release component.

In another embodiment of the above aspect, the said modified-release composition comprises an extended-release component and a delayed-release component.

In another embodiment of the above aspect, the said modified-release oral pharmaceutical composition comprises a combination of an extended release component, a delayed-release component, and an immediate-release component.

In a further embodiment, the said extended release component may be gastro retentive and comprise gastro-retentive component for example effervescent producing ingredient or viscosity enhancing agent and combination thereof.

In another embodiment of the above aspect, the ratio of isotretinoin in the immediate-release and modified-release component is about 1:99 to about 99:1.

In yet another embodiment of the above aspect, the ratio isotretinoin in the immediate-release and extended-release component is about 10:90 to about 70:30.

In another embodiment of the above aspect, the oral pharmaceutical composition comprises:
(a) 1 mg to 80 mg of isotretinoin or a pharmaceutically acceptable salt, ester or a derivative thereof; and
(b) a release modifying agent.

In another embodiment of the above aspect, the oral pharmaceutical composition comprises:
(a) 1 mg to 20 mg of isotretinoin or a pharmaceutically acceptable salt, ester or a derivative thereof; and
(b) a release modifying agent.

In another embodiment of the above aspect, the oral pharmaceutical composition comprises isotretinoin in an amount of about 4 mg to about 100 mg, about 8 mg to about 92 mg, about 10 mg to about 90 mg, about 12 mg to about 88 mg, or about 20 mg to about 80 mg.

In another embodiment of the above aspect, the modified-release oral pharmaceutical composition is in the form of solid or liquid composition. Solid composition includes tablet or capsule dosage form. Tablet dosage form includes matrix type composition, a reservoir type composition, a layered composition or inlay tablets.

In another embodiment of the above aspect, the modified-release oral pharmaceutical composition is in the form of a capsule, wherein the capsule shell is coated with the coating of a release modifying agent.

In another embodiment of the above aspect, the oral pharmaceutical composition is in the form of a capsule, wherein the capsule fill comprises one or more release modifying agents.

In another embodiment of the above aspect, the oral pharmaceutical composition is in the form of a capsule, wherein the capsule fill comprises solid dispersion of isotretinoin.

In another embodiment of the above aspect, the oral pharmaceutical composition is in the form of a capsule, wherein the capsule fill comprises solid dispersion of isotretinoin distributed uniformly with one or more release modifying agents.

In another embodiment of the above aspect, the oral pharmaceutical composition is in the form of a capsule, wherein the capsule fill comprises spray dried blend of isotretinoin distributed uniformly with one or more release modifying agents.

In yet another embodiment of the above aspect, the oral pharmaceutical composition is in the form of a capsule, wherein the capsule comprises immediate-release component and extended-release components to be filled into capsules.

In yet another embodiment of the above aspect, the oral pharmaceutical composition is in the form of a capsule, wherein the capsule comprises immediate-release tablet and extended-release tablet to be filled into capsules.

In yet another embodiment of the above aspect, the oral pharmaceutical composition is in the form of a capsule, wherein the capsule comprises an extended-release capsule and an immediate-release liquid to be filled into capsules.

In yet another embodiment of the above aspect, the oral pharmaceutical composition is in the form of a capsule, wherein the ratio of isotretinoin to a release modifying polymer in the composition ranges from about 1:1 to about 1:40. In particular, the ratio of isotretinoin to release modifying polymer in the composition is about 1:2 to about 1:20.

In yet another embodiment of the above aspect, the isotretinoin is about 2% to about 40% by weight of the immediate-release component.

In yet another embodiment of the above aspect, the isotretinoin is about 3% to about 30% by weight of the immediate-release component.

In yet another embodiment of the above aspect, the isotretinoin is about 0.5% to about 20% by weight of the extended-release component.

In yet another embodiment of the above aspect, the isotretinoin is about 1% to about 15% by weight of the extended-release component.

In another embodiment of the above aspect, the oral pharmaceutical composition is in the form of a capsule, wherein the capsule fill comprises a combination of immediate-release, extended-release and delayed-release components.

In yet another embodiment of the above aspect, the ratio of the immediate-release, extended-release and delayed-release components is about 40:40:20.

In yet another embodiment of the present disclosure, the modified-release tablet composition may comprise one or more gastro-retention providing components.

In yet another embodiment of the above aspect, the gastro-retentive component as described above further provide buoyancy, providing extended-release of isotretinoin from the capsule.

In yet another embodiment of the present disclosure, the immediate-release, modified-release, and delayed-release components described above are prepared from solid dispersion particles comprising isotretinoin.

In yet another embodiment of the present disclosure, the immediate-release, modified-release, and delayed-release tablet components described above are prepared from spray-dried, solid dispersion particles comprising isotretinoin.

In yet another embodiment of the above aspect, the delayed-release tablet component is prepared by coating a part of an immediate-release core comprising spray-dried blend of isotretinoin with that of a delayed-release polymer.

In yet another embodiment of the above aspect, the ratio of isotretinoin in the immediate-release component and extended-release component is about 30:70 to about 60:40.

In yet another embodiment of the above aspect, the oral pharmaceutical composition exhibits reduced food effect as indicated by comparable Cₘₐₓ and AUC in fasting and fed states.

In yet another embodiment of the above aspect, the modified-release, oral pharmaceutical exhibits steady state ratio of Cₘₐₓ to Cₙᵤₙ in the range of about 6:1 to 1.25:1, when administered to human subject once daily in fed state. In particularly, ratio of Cₘₐₓ to Cₙᵤₙ in the range of about 2.5:1 to 5:1 when administered to human subject once daily in fed state.

The term “Cmin” is the minimum drug concentration in plasma or serum. In some embodiments, “Cmin” refers to the minimum drug concentration in plasma or serum before the next dose is administered. The term “Cmax” is the maximum drug concentration in plasma or serum. The term “Tmax” refers to the time required to reach Cmax.

In yet another embodiment of the above aspect, the modified-release, oral pharmaceutical composition provides the same therapeutic effect in comparison to the already marketed formulation of isotretinoin.

In yet another embodiment of the above aspect, the oral pharmaceutical composition comprises isotretinoin, wherein the particle size distribution of isotretinoin is such that the D₉₀ of the particles is less than 60 μm, less than 55 μm, less than 50 μm, or less than 45 μm.

In another embodiment of the above aspect, the oral pharmaceutical composition comprises isotretinoin, wherein the particle size distribution of isotretinoin is such that the D₉₀ of the particles is less than 60 μm, less than 40 μm, less than 35 μm, or less than 25 μm. In another embodiment of the above aspect, the oral pharmaceutical composition comprises isotretinoin, wherein the particle size distribution of isotretinoin is such that the D₉₀ of the particles is about 60 μm to about 10 μm, about 40 μm to about 10 μm, about 35 μm to about 10 μm, or about 25 μm to about 10 μm.

In another embodiment of the above aspect, the oral pharmaceutical composition comprises isotretinoin, wherein the particle size distribution of isotretinoin is such that the D₉₀ of the particles is less than 50 μm, less than 30 μm, less than 20 μm, or less than 10 μm. In another embodiment of the above aspect, the oral pharmaceutical composition comprises isotretinoin, wherein the particle size distribution of isotretinoin is such that the D₉₀ of the particles is about 50 μm to about 1 μm, about 30 μm to about 1 μm.

In yet another embodiment of the above aspect, the oral pharmaceutical composition comprises isotretinoin, wherein the particle size distribution of isotretinoin is such that the D₅₀ of the particles is less than 40 μm, less than 35 μm, less than 30 μm, or less than 25 μm. In yet another embodiment of the above aspect, the oral pharmaceutical composition comprises isotretinoin, wherein the particle size distribution of isotretinoin is such that the D₅₀ of the particles is about 40 μm to about 1 μm, about 35 μm to about 1 μm, about 30 μm to about 1 μm, or about 25 μm to about 1 μm.

In yet another embodiment of the above aspect, the oral pharmaceutical composition comprises isotretinoin, wherein the particle size distribution of isotretinoin is such that the D₅₀ of the particles is less than 40 μm, less than 30 μm, less than 20 μm, or less than 15 μm. In yet another embodiment of the above aspect, the oral pharmaceutical composition comprises isotretinoin, wherein the particle size distribution of isotretinoin is such that the D₅₀ of the particles is about 40 μm to about 1 μm, about 30 μm to about 1 μm, about 20 μm to about 1 μm, or about 15 μm to about 1 μm.

In another embodiment of the above aspect, the oral pharmaceutical composition comprises isotretinoin, wherein the particle size distribution of isotretinoin is such that the D₁₀ of the particles is less than 20 μm, less than 17 μm, less than 15 μm, or less than 12 μm.

In another embodiment of the above aspect, the oral pharmaceutical composition comprises isotretinoin, wherein the particle size distribution of isotretinoin is such that the D₁₀ of the particles is less than 18 μm, less than 15 μm, less than 10 μm, or less than 8μm.

In another embodiment of the above aspect, the oral pharmaceutical composition comprises isotretinoin, wherein the particle size distribution of isotretinoin is such that the D₁₀ of the particles is less than 10 μm, less than 7 μm, less than 5 μm, or less than 2 μm.

In yet another embodiment of the above aspect, the oral pharmaceutical composition comprises isotretinoin, wherein the particle size distribution of isotretinoin is such that the D₉₀ of the particles is less than 60 μm and the D₅₀ of the particles is less than 40 μm.

In yet another embodiment of the above aspect, the oral pharmaceutical composition comprises isotretinoin, wherein the particle size distribution of isotretinoin is such that the D₉₀ of the particles is less than 60 μm, the D₅₀ of the particles is less than 40 μm and the D₁₀ of the particles is less than 20 μm.

The size reduction of isotretinoin is achieved by wet milling the solid dispersion of isotretinoin in an oily vehicle or the dispersion of isotretinoin in an aqueous medium using mechanical means such as a ball mill, and media mills such as a sand mill, Dyno®-Mill (Glen Mills, Inc, Clifton N.J.), or bead mill. The grinding media in these mills can comprise spherical particles, such as stainless steel beads or zirconium oxide balls. The size reduction of isotretinoin can also be achieved by spray drying the solution of isotretinoin in a non-aqueous vehicle.

The term “solid dispersion” refers to a solidified form of a drug obtained by dispersing or dissolving the drug in a matrix. In the solid dispersion the drug is present in a molecular state, colloidal state, metastable state, or an amorphous state. The one or more additional pharmaceutically acceptable excipients can be selected from binders, disintegrants, fillers, lubricants, glidants, surfactants, stabilizing agents, antioxidants, alkaline stabilizers, colors, flavors, preservatives, and combinations thereof.

In another aspect of the present disclosure, there is provided a process for preparing said solid dispersion, wherein the process is a solvent evaporation method, melting method, kneading method, co-grinding method, co-precipitation method, freeze-drying, spray-drying, coating, or adsorbing the drug onto carrier particles. The percentage of isotretinoin in the solid dispersion ranges from about 25% to about 60% based on the total weight of solid dispersion.

The term “solvent” as used herein refers to any solvent or solvent mixture, aqueous and non-aqueous solvents, protic or aprotic solvents; for example, water, alcohols, esters, halogenated hydrocarbons, ketones, ethers, and mixtures thereof. Examples of alcohols include, primary, secondary and tertiary alcohols, for example methanol, ethanol, n-propanol, isopropanol, and butanol. The esters may include one or more of ethyl acetate, n-propyl acetate, isopropyl acetate, and n-butyl acetate. Examples of halogenated hydrocarbons include dichloromethane, chloroform, and 1,2-dichloroethane. Examples of ketones include acetone, methyl ethyl ketone, and the like. In particular, the solvent used are the mixture of the ratio of dichloromethane and ethanol in the range of about 9:1 to about 1:9.

In another embodiment of the above aspect, the oral pharmaceutical composition comprises isotretinoin in a dissolved form.

In another embodiment of the above aspect, the release modifying agent comprises a release modifying polymer, a lipidic material, a polysaccharide, and mixtures thereof.

In another embodiment of the above aspect, the release modifying polymer is present in an amount of about 1% w/w to about 90% w/w of the total composition, or in an amount of about 15% w/w to about 80% w/w of the total composition, or in an amount of about 5% w/w to about 50% w/w of the total composition.

In yet another embodiment of the above aspect, the release modifying polymer is present in an amount ranging from about 15% to about 80% by weight of the total weight of capsule, wherein capsule shell weight is excluded.

In yet another embodiment of the above aspect, the extended release component or immediate release component or delayed release component is in the form of a solution, suspension, emulsion, nanoemulsion, solid particles adsorbed onto carrier substrate, spray-dried particles, dispersion, solid dispersion, powder, granules, pellets, minitablets, microcapsules, spheroids, capsules, or tablets.

In yet another embodiment, the said oral pharmaceutical composition is stable when stored at 40° C. and 75% relative humidity, or at 25° C. and 60% relative humidity, for a period of at least three months or to the extent necessary for use of the composition.

In another aspect, the present disclosure provides a process for preparing an oral pharmaceutical composition comprising isotretinoin suitable for once-daily administration wherein said process comprises wet or dry granulation using a fluidized bed granulator or high shear mixer granulator; direct compression; extrusion-spheronization; melt granulation/extrusion; spray-drying; spray-congealing; freeze-drying; or any other conventional process known in the art.

In one embodiment of the above aspect, the isotretinoin is homogenously dispersed throughout the pharmaceutical composition.

In another aspect, the present disclosure provides a process for preparing a modified release component comprising:
(a) 1 mg to 80 mg of isotretinoin or a pharmaceutically acceptable salt, ester, or a derivative thereof;
(b) a release modifying agent; and
(c) a pharmaceutically acceptable excipient,
   wherein the said process comprises wet granulation using a fluidized bed granulator or high shear mixer granulator; dry granulation, direct compression; extrusion-spheronization; melt granulation/extrusion; spray-drying; spray-congealing; freeze-drying; or any other conventional process known in the art.

In another aspect of the present disclosure, there is provided a process for the preparation of modified-release, once-daily, oral pharmaceutical composition comprising isotretinoin; wherein the process comprises:
(a) preparing finely milled spray-dried particles of isotretinoin solid dispersion;
(b) adding weighed quantity of spray dried blend of step (a) with suitable excipients to form an immediate-release tablet component;
(c) adding weighed quantity of spray dried blend of step (a) with suitable excipients to form a modified-release tablet component; and
(d) filling of tablet components of step (b) and step (c) into a capsule.

In still another aspect, the present disclosure provides a method of treating acne, musculoskeletal and connective tissue inflammations, emphysema, ulcerating diseases, cervical tumors in HIV positive women, lung cancer in smokers, skin cancer, neuroblastoma, recurrent prostate cancer, leukemia, high-grade glioma, head and neck cancers, multiple myeloma, gram-negative folliculitis, recalcitrant rosacea, pyoderma faciale, psoriasis, cutaneous lupus erythematosus, acne fulminans, squamous cell carcinoma, cutaneous photoaging, and other off-label indications of isotretinoin by administering to an individual in need thereof an oral pharmaceutical composition of the present disclosure once-daily.

In one embodiment of the above aspect, the present disclosure provides a method of treating acne by administering to an individual in need thereof a modified-release, oral pharmaceutical composition of the present disclosure once-daily.

In another aspect, the present disclosure provides a method of treating acne, wherein the said method comprises administering to an individual in need thereof about 0.4 mg/kg to about 2.4 mg/kg of isotretinoin in an oral dosage form once-daily, wherein the therapeutic blood plasma levels of isotretinoin are maintained approximately over a twenty-four hour period.

In one embodiment of the above aspect, the present disclosure provides a method of treating acne, wherein said method comprises administering to an individual in need thereof about 0.45 mg/kg to about 2.2 mg/kg of isotretinoin in an oral dosage form once-daily, wherein the therapeutic blood plasma levels of isotretinoin are maintained approximately over a twenty-four hour period.

In another embodiment of the above aspect, the present disclosure provides a method of treating acne, wherein said method comprises administering to an individual in need thereof about 0.5 mg/kg to about 2 mg/kg of isotretinoin in an oral dosage form once-daily, wherein the therapeutic blood plasma levels of isotretinoin are maintained approximately over a twenty-four hour period.

In another embodiment of the above aspect, the present disclosure provides a method for treating acne, wherein the dose of isotretinoin is about 16 mg to about 240 mg, or about 18 mg to about 220 mg, or about 20 mg to about 200 mg once-daily.

In yet another embodiment of the above aspect, the modified-release, oral pharmaceutical composition comprising isotretinoin suitable for once-daily administration, wherein the composition releases not less than 25% of isotretinoin in 4 hours, when measured in United States Pharmacopeia (USP) type II dissolution apparatus, paddle at 75 rpm, in 900 mL of pH 8.0 borate buffer, containing 0.5% cetrimide and 50 mg per L of pancreatin as dissolution media with alternate sinkers.

In yet another embodiment of the above aspect, the modified-release, oral pharmaceutical composition, wherein the composition releases not less than 50% of isotretinoin in 12 hours, when measured in United States Pharmacopeia (USP) type II dissolution apparatus, paddle at 75 rpm, in 900 mL of pH 8.0 borate buffer, containing 0.5% cetrimide and 50 mg per L of pancreatin as dissolution media with alternate sinkers.

In another aspect, the present disclosure provides a stable oral pharmaceutical composition comprising isotretinoin and a pharmaceutically acceptable excipient wherein the composition contains not more than 1.5% w/w 5, 6-epoxy-13-cis retinoic acid when stored at a temperature 40° C. and 75% relative humidity for a period of three months.

In some embodiments, the compositions described herein are suitable for once daily administration. In some embodiments, the phrase “suitable for once daily administration” refers to a composition in which the desired therapeutic effect is achieved by administering the composition once daily. In some embodiments, the phrase “suitable for once daily administration” refers to a composition in which a therapeutic effect achieved upon daily administration is similar to the therapeutic effect achieved by twice daily administration of marketed capsules, for example, twice daily administration of Absorica® (Sun Pharmaceuticals Industries, India). In some embodiments, “suitable for once daily administration” refers to a composition in which the therapeutic blood plasma levels of isotretinoin are maintained over about a twenty-four hour period when administered once daily. In some embodiments, the phrase “suitable for once daily administration” refers to a composition in which a therapeutic effect achieved by once daily administration of amount of 0.5 to 1.0 mg/kg/day isotretinoin is similar to the therapeutic effect achieved by twice daily administration of marketed capsules. In some embodiments, the term “suitable for once daily administration” refers to composition having at least one dissolution profile, at least one release profile, or at least one pharmacokinetic profile as described herein.

The phrase “modified-release” as used herein relates to a kind of release pattern wherein the active pharmaceutical ingredient is released from the composition over an extended period of time, and encompasses prolonged, osmotic, sustained, controlled, chrono-therapeutic, pH-dependent, extended, pulsed, and delayed-release, or combinations thereof.

The “modified-release component” or “immediate release component” as used herein can be in the form of solution, suspension, emulsion, nanoemulsion, solid particles adsorbed onto carrier substrate, spray-dried particle dispersion, solid dispersion, powder, granules, pellets, minitablets, microcapsules, spheroids, capsules, tablets (including matrix, reservoir, inlay), or any other suitable dosage form.

Matrix-type compositions are those in which the drug is distributed uniformly in the matrix of one or more release modifying agents, and reservoir type compositions utilize coating of release modifying agent over the core comprising the drug.

Multiple unit particulate systems are single-unit formulations that contain the active ingredient within the single tablet or capsule, whereas multiple-unit dosage forms comprise a number of discrete particles that are combined into one dosage unit. These discrete units may be granules, pellets, spheroids, tablets, or beads. These discrete units have individual release patterns.

A pH-dependent system may contain four different tablets - one providing immediate-release, a second providing release at pH 5.5, a third providing release at pH 6, and a fourth providing release at pH 7, wherein the tablets are filled into a capsule to provide a single unit system.

A gastro retentive system is a system which is designed to retain in the stomach for a prolonged time and release the active pharmaceutical ingredient, thereby enabling sustained and prolonged availability of the drug to the upper part of the gastrointestinal (GI) tract. Different approaches or systems to prolong the gastric residence time include mucoadhesive or bio-adhesive systems, high density systems, expandable or swelling systems, and floating drug delivery systems, including gas generating systems.

The phrase “release modifying agent” as used herein refers to an agent or material which helps in achieving the desired modified-release of the composition and includes one or more of a release modifying polymer, a lipidic material, a polysaccharide, and mixtures thereof.

A release modifying polymer may be selected from hydrophilic polymers which include both water soluble and water swellable polymers, hydrophobic polymers, enteric polymers, delayed-release polymers, and mixtures thereof. The ratio isotretinoin to release modifying polymer is from about 1:1 to about 1:40.

Examples of hydrophilic polymers include, but are not limited to, hydroxypropylmethyl cellulose, hydroxypropyl cellulose, hydroxyethyl cellulose, hydroxymethyl cellulose, carboxymethyl cellulose, sodium carboxymethyl cellulose, carboxymethyl cellulose calcium, polyvinylpyrrolidone, polyethylene oxide, polyvinyl alcohol, and mixtures thereof. Additionally, hydrophilic polymers include but are not limited to, ammonium alginate, sodium alginate, potassium alginate, calcium alginate, propylene glycol alginate, alginic acid, xanthan gum, guar gum, locust bean gum, potassium pectate, potassium pectinate, and derivatives and mixtures thereof.

Examples of hydrophobic polymers include, but are not limited to, methyl cellulose, ethyl cellulose, propyl cellulose, ethyl methyl cellulose, isopropyl cellulose, ethyl propyl cellulose, butyl cellulose, benzyl cellulose; cellulose esters such as cellulose acetate, cellulose butyrate, cellulose propionate, cellulose butyrate, and cellulose acetate propionate; cellulose cyanoalkyl ethers such as cyanoethyl cellulose, cyanomethyl cellulose, cyanoethylmethyl cellulose, cyanopropyl cellulose, methacrylic acid-acrylic acid copolymers (e.g., Eudragit® RS (Evonik Healthcare, Germany), Eudragit® RL, Eudragit® NE, Eudragit® RSPO, and Eudragit® RLPO) and mixtures thereof.

Examples of enteric polymers or delayed-release polymers include, but are not limited to, methacrylic acid copolymers, ammonio methacrylate copolymer, hydroxypropylmethyl cellulose phthalate, hydroxypropylmethyl cellulose acetate succinate, cellulose acetate phthalate, and mixtures thereof.

Examples of lipidic materials include, but are not limited to, vegetable oils or hydrogenated vegetable oils such as soyabean oil, cottonseed oil, peanut oil, castor oil, sesame oil, and Kolliwax® (Hydrogenated castor oil; BASF, Germany); waxes such as beeswax, carnauba wax, microcrystalline wax, candelilla wax, lecithin, paraffin wax, shellac wax, and petrolatum; fatty acids such as stearic acid, capric, caproic acid, linoleic acid, linolenic acid, palmitic acid, lauric acid, and eleostearic acid; fatty alcohols such as lauryl alcohol, cetostearyl alcohol, stearyl alcohol, cetyl alcohol, and myristyl alcohol; fatty acid esters such as glycerol monostearate, glycerol monooleate, acetylated monoglycerides, tristearin, tripalmitin, and cetyl esters glyceryl palmitostearate; glyceryl behenate; medium chain triglycerides such as Neobee® 0 and Neobee® M5, Miglyol® 629, 810, 812, 818 and Miglyol® 829 (Caprylic/Capric/Succinic Triglyceride), Captex® 350, 355 and 810D, Labrafac™ lipophile WL 1349, Crodamol™ GTCC, Gelucire®, such as glyceryl palmitostearate (Precirol®), glycerol esters of fatty acids (Gelucire® 43/01, Gelucire® 39/01, and Gelucire® 50/13 (Stearoyl Macrogol-32 glycerides)), polyethyleneglycol derivatives, and mixtures thereof.

Examples of oily vehicles include, but are not limited to, fatty acid esters, fatty acids, fatty alcohols, vegetable oils, and mixtures thereof. Examples of suitable fatty acid esters include polyol esters of medium chain fatty acids selected from esters and mixed esters of glycerol, propylene glycol, polyglycerol, polyethylene glycol with medium chain fatty acids, phosphatidyl choline with medium chain glycerides, and mixtures thereof. Examples of suitable fatty acids include C₆-C₂₀ saturated or mono- or di-unsaturated acid, for example, oleic acid, linoleic acid, caprylic acid, and caproic acid. Examples of suitable fatty alcohols include C₆-C₂₀ saturated or mono- or di-unsaturated alcohols, for example, oleyl alcohol, capryl alcohol and capric alcohol. Examples of suitable vegetable oils include groundnut oil, olive oil, soybean oil, safflower oil, sunflower oil, palm oil, sesame oil, canola oil, and corn oil. The oily vehicle may comprise from about 5% w/w to about 95% w/w of the total composition.

Examples of surfactants or surface stabilizers include, but are not limited to, sorbitan monostearate; polysorbates prepared from lauric, palmitic, stearic, and oleic acid (e.g., polysorbate 80); polyoxyethylene monoesters such as polyoxyethyl ethylene monostearate, polyoxyethylene monolaurate, and polyoxyethylene monooleate; dioctyl sodium sulfosuccinate; sodium lauryl sulfate; and poloxamers. Lecithin, gelatin, casein, gum acacia, stearic acid, calcium stearate, glycerol monostearate, sorbitan esters, macrogol ethers such as cetomacrogol 1000, polyoxyethylene castor oil derivatives, polyoxyethylene sorbitan fatty acid esters such as Tweens, polyoxyethylene stearates, colloidal silicon dioxide (Aerosil®), sodium dodecylsulfate, carboxymethylcellulose calcium, carboxymethylcellulose sodium, hydroxyethylcellulose, hydroxypropylcellulose, hydroxypropyl methylcellulose, polyvinylpyrrolidone (PVP), poloxamers such as Pluronics® F68 and F108, dioctyl sodium sulfosuccinate (DOSS), docusate sodium, sodium lauryl sulfate, Span® 20 and 80, Campul® PG8 (Propylene Glycol Monocaprylate), and macrogolglycerol esters such as Cremophor® EL, Polyethoxylated castor oil, or Polyoxyl 40 Hydrogenated Castor Oil.

Examples of effervescence producing ingredients include, but not limited to, citric acid, tartaric acid, ascorbic acid, fumaric acid, malic acid, adipic acid, succinic acid, nicotinic acid, sodium alginate, sodium dihydrogen phosphate, disodium dihydrogen pyrophosphate, acid citrate salts, sodium acid sulphate, sodium bicarbonate, sodium carbonate, potassium carbonate, potassium bicarbonate, sodium sesquicarbonate, sodium glycine carbonate, lysine carbonate, arginine carbonate, calcium carbonate, and mixtures thereof. The ratio of isotretinoin to effervescent excipient is from about 1:0.05 to about 1:10. In particular, about 1:0.5 to about 1:6.

Viscosity enhancing agents, but are not limited to, ammonium alginate, sodium alginate, potassium alginate, calcium alginate, propylene glycol alginate, alginic acid, xanthan gum, guar gum, locust bean gum, potassium pectate, potassium pectinate, and derivatives and mixtures thereof.

Examples of alkaline stabilizers include, but are not limited to, primary, secondary, and tertiary amines, cyclic amines, N,N′-dibenzylethylenediamine, diethanolamine, ethylenediamine, meglumine (N-methylglucamine), monosodium glutamate, polacrillin sodium, sodium alginate, and mixtures thereof. The ratio of isotretinoin to meglumine is from about 1:0.01 to about 1:1.

Examples of antioxidants include, but are not limited to, butylated hydroxyl anisole, butylated hydroxyl toluene, tocopherol, ascorbyl palmitate, ascorbic acid, sodium metabisulfite, sodium sulfite, sodium thiosulfate, propyl gallate, and mixtures thereof.

Examples of carrier substrates used for adsorption of the drug include but are not limited to lactose; microcrystalline cellulose; calcium phosphate; hydroxyl propyl methyl cellulose, dextrin; dextrose; sucrose; mannitol; maltodextrin; sodium alumino silicate; clays, including bentonite, kaolin, montmorrillonite, attapulgite, halloysite, laponite, and the like; silica, including colloidal silica, mesoporous silica, and fumed silica; zeolites; talc; cholesteramine; polystyrene sulfonates; mono and polysulfonated resins; activated charcoal; magnesium aluminometasilicate; Neusilin®; and mixtures thereof.

Examples of suitable preservatives include, but are not limited to, methyl paraben, ethyl paraben, propyl paraben, butyl paraben, benzoic acid, sodium benzoate, benzyl alcohol, sorbic acid, potassium sorbate, and mixtures thereof.

The oral pharmaceutical composition of the present disclosure can further comprise one or more pharmaceutically acceptable excipients selected from one or more of fillers, binders, osmogens, disintegrants, lubricants, anti-adherents, glidants, solvents, coloring agents, and flavoring agents.

Examples of fillers or diluents include, but are not limited to, lactose, anhydrous lactose, sorbitol, calcium dihydrogen phosphate dihydrate, calcium phosphate-dibasic, calcium phosphate-tribasic, calcium sulfate, microcrystalline cellulose, silicified microcrystalline cellulose, mannitol, starch, pregelatinized starch, and mixtures thereof.

Examples of binders include, but are not limited to, corn starch, pregelatinized starch, microcrystalline cellulose, silicified MCC (e.g., Prosolv® HD 90), methyl cellulose, hydroxypropyl cellulose (HPC-L), methylcellulose, carboxymethyl cellulose sodium, hydroxypropyl methylcellulose, polyvinylpyrrolidone, and mixtures thereof.

Examples of disintegrants include, but are not limited to, cross-linked polyvinyl pyrrolidone, corn starch, modified starches, agar-agar, calcium carbonate, sodium carbonate, alginic acids, cross-carmellose sodium, sodium starch glycolate, microcrystalline cellulose, hydroxypropyl cellulose (L-HPC), and mixtures thereof.

Examples of anti-adherents include, but are not limited to, magnesium stearate, talc, calcium stearate, glyceryl behenate, stearic acid, and mixtures thereof.

Examples of lubricants and glidants include, but are not limited to, colloidal anhydrous silica, stearic acid, magnesium stearate, calcium stearate, talc, microcrystalline wax, yellow beeswax, white beeswax, sodium stearyl fumarate, and mixtures thereof.

Solvents include aqueous or non-aqueous solvents.

The coloring agents and flavoring agents may be selected from any FDA approved colors or flavors for oral use.

The coating layer present can further comprise other excipients, such as film forming polymers, solvents, plasticizers (e.g., triethyl citrate, triacetin, Myvacet®), antiadherents, and opacifiers, and optionally colorants and polishing agents.

The term “about” as used herein, refers to any value which lies within the range defined by a variation of up to±10% of the value.

The term “food effect” as used herein means food-drug interactions which either decrease or increase the extent of drug absorption. It refers to a relative difference in AUC, Cₘₐₓ, and/or tₘₐₓ of a drug, when said drug or a formulation thereof is administered orally to a human concomitantly with food or in a fed state as compared to the same values when the same formulation is administered in a fasted state or without food.

The phrase “homogenous” as used herein, means that the active ingredients are substantially evenly dispersed throughout the part of the finished tablet which comprises them. The homogeneity of active ingredients in a tablet may be ascertained by means of the drug uniformity measure.

The following examples represent various embodiments according to the present disclosure. The examples are given solely for the purpose of illustration and are not to be construed as limitations of the present disclosure, as many variations thereof are possible without departing from the spirit and scope of the disclosure.

### EXAMPLES

### Example 1

| | | |
|---|---|---|
| S. No. | Ingredients | Quantity (% w/w) |

Core Capsule

| | | |
|---|---|---|
| 1. | Isotretinoin | 13.92 |
| 2. | Butylated hydroxy anisole | 3.83 |
| 3. | Polysorbate 80 | 0.08 |
| 4. | Soyabean oil | 82.17 |
| 5. | Gelatin | q.s. |
| 6. | Purified water | q.s. |
| 7. | FDA approved color | q.s. |

Capsule coating

| | | |
|---|---|---|
| 8. | Ethyl cellulose | 38.15 |
| 9. | Hydroxypropylmethyl cellulose | 57.25 |
| 10. | Triethyl citrate | 4.60 |
| 11. | Isopropyl alcohol | q.s |
| 12. | Purified water | q.s. |

### Procedure:

1. Butylated hydroxy anisole and polysorbate 80 were dissolved in soyabean oil to form a clear solution.

2. Isotretinoin was added to the solution of step 1 under stirring to obtain a uniform dispersion.

3. The dispersion of step 2 was milled to get a particle size of isotretinoin such that the D₉₀ was about 21 μm.

4. The dispersion of step 3 was filled into hard gelatin capsules.

5. Gelatin was soaked in purified water at a temperature of 60° C. to 90° C. along with color to get a uniform solution.

6. The filled capsules of step 4 were band-sealed using the gelatin solution of step 5.

7. Ethyl cellulose was dissolved in isopropyl alcohol under stirring.

8. Hydroxypropylmethyl cellulose was dissolved in water, and this aqueous solution was added to the solution of step 7 under stirring.

9. Triethyl citrate was added in solution of step 8 and stirring was continued for a few minutes to ensure homogeneity of the coating solution.

10. The filled capsules of step 6 were coated with the coating solution of step 9.

### Example 2

| S. No. | Ingredients | Quantity (% w/w) |
|---|---|---|
| 1. | Isotretinoin | 6.25 |
| 2. | Butylated hydroxy anisole | 1.72 |
| 3. | Polysorbate 80 | 0.04 |
| 4. | Soyabean oil | 85.74 |
| 5. | Hydroxypropylmethyl cellulose | 6.25 |
| 6. | Gelatin | q.s. |
| 7. | Purified water | q.s. |
| 8. | Sunset yellow color | q.s. |

### Procedure

1. Butylated hydroxy anisole and polysorbate 80 were dissolved in soyabean oil (43% w/v) to form a clear solution.

2. Isotretinoin was added to the solution of step 1 under stirring to obtain a uniform dispersion.

3. The dispersion of step 2 was milled to get a particle size of isotretinoin such that the D₉₀ was about 15 μm.

4. Hydroxypropylmethyl cellulose was added to the remaining quantity of soyabean oil (57% w/v) under stirring to obtain a uniform dispersion.

5. The dispersion of step 3 was added into the dispersion of step 4 under stirring to obtain a uniform dispersion.

6. The dispersion of step 5 was filled into hard gelatin capsules.

7. Gelatin was soaked in purified water at a temperature of 60° C. to 90° C. along with idacol sunset yellow color to get a uniform solution.

8. The filled capsules of step 6 were band-sealed using the gelatin solution of step 7.

### Example 3

| S. No. | Ingredients | Quantity (% w/w) |
|---|---|---|
| 1. | Isotretinoin | 6.35 |
| 2. | Butylated hydroxy anisole | 1.75 |
| 3. | Polysorbate 80 | 0.04 |
| 4. | Soyabean oil | 87.10 |
| 5. | Gelucire ® 43/01 | 4.76 |
| 6. | Gelatin | q.s. |
| 7. | Purified water | q.s. |
| 8. | Sunset yellow color | q.s. |

### Procedure

1. Butylated hydroxy anisole and polysorbate 80 were dissolved in soyabean oil (43% w/w) to form a clear solution.

2. Isotretinoin was added to the solution of step 1 under stirring to obtain a uniform dispersion.

3. The dispersion of step 2 was milled to get a particle size of isotretinoin such that the D₉₀ was about 15 μm.

4. Gelucire® 43/01 was melted at 55° C.

5. The remaining quantity of soyabean oil (57% w/w) was heated at 55° C. and mixed with the product of step 4 under stirring,

6. The dispersion of step 3 was added into the mixture of step 5 under stirring to obtain a uniform dispersion.

7. The dispersion of step 6 was filled into hard gelatin capsules.

8. Gelatin was soaked in purified water at a temperature of 60° C. to 90° C. along with idacol sunset yellow color to get a uniform solution.

9. The filled capsules of step 7 were band-sealed using the gelatin solution of step 8.

### Example 4

| S. No. | Ingredients | Quantity (% w/w) |
|---|---|---|
| 1. | Isotretinoin | 6.35 |
| 2. | Butylated hydroxy anisole | 1.75 |
| 3. | Polysorbate 80 | 0.04 |
| 4. | Soyabean oil | 87.10 |
| 5. | Glycerol monostearate | 4.76 |
| 6. | Gelatin | q.s. |
| 7. | Purified water | q.s. |
| 8. | Sunset yellow color | q.s. |

### Procedure

1. Butylated hydroxy anisole and polysorbate 80 were dissolved in soyabean oil (43% w/w) to form a clear solution.

2. Isotretinoin was added to the solution of step 1 under stirring to obtain a uniform dispersion.

3. The dispersion of step 2 was milled to get a particle size of isotretinoin such that the D₉₀ was about 15 μm.

4. Glycerol monostearate was melted at 65° C.

5. The remaining quantity of soyabean oil (57% w/w) was heated at 65° C. and mixed with the product of step 4 under stirring.

6. The dispersion of step 3 was added into the mixture of step 5 under stirring to obtain a uniform dispersion.

7. The dispersion of step 6 was filled into hard gelatin capsules.

8. Gelatin was soaked in purified water at 60° C. to 90° C. along with idacol sunset yellow color to obtain a uniform solution.

9. The filled capsules of step 7 were band-sealed using the gelatin solution of step 8.

### Example 5

| S. No. | Ingredients | Quantity (% w/w) |
|---|---|---|
| 1. | Isotretinoin | 6.25 |
| 2. | Butylated hydroxy anisole | 1.72 |
| 3. | Polysorbate 80 | 0.04 |
| 4. | Soyabean oil | 52.54 |
| 5. | Eudragit | 7.81 |
| 6. | Caprylic/Capric/Succinic Triglyceride | 31.64 |
| 7. | Gelatin | q.s. |
| 8. | Purified water | q.s. |
| 9. | Sunset yellow color | q.s. |

### Procedure

Butylated hydroxy anisole and polysorbate 80 were dissolved in soyabean oil (70% w/v) to form a clear solution.

Isotretinoin was added to the solution of step 1 under stirring to obtain a uniform dispersion.

The dispersion of step 2 was milled to get a particle size of isotretinoin such that the D90 was about 15 μm.

Eudragit was added to Caprylic/Capric/Succinic Triglyceride under stirring.

The remaining quantity of soyabean oil (30% w/v) was added to the dispersion of step 4 under stirring.

The dispersion of step 3 was added into the dispersion of step 5 under stirring to obtain a uniform dispersion.

The dispersion of step 6 was filled into hard gelatin capsules.

Gelatin was soaked in purified water at a 60° C. to 90° C. along with idacol sunset yellow color to obtain a uniform solution.

The filled capsules of step 7 were band-sealed using the gelatin solution of step 8.

### Example 6

| S. No. | Ingredients | Quantity (% w/w) |
|---|---|---|
| 1. | Isotretinoin | 10.78 |
| 2. | Lactose anhydrous | 64.69 |
| 3. | Butylated hydroxy anisole | 0.27 |
| 4. | Povidone | 5.39 |
| 5. | Polyoxyl 40 Hydrogenated Castor Oil | 5.39 |
| 6. | Hydroxypropylmethyl cellulose | 8.09 |
| 7. | L-Hydroxypropyl cellulose | 5.39 |
| 8. | Dichloromethane | q.s. |
| 9. | Ethanol | q.s. |

### Procedure

1. Povidone, Polyoxyl 40 Hydrogenated Castor Oil and hydroxypropylmethyl cellulose K100M were added to a mixture of dichloromethane and ethanol (in a ratio of 70:30) under stirring to form a clear solution.

2. Butylated hydroxy anisole and isotretinoin were dissolved in the solution of step 1.

3. Lactose and L-hydroxypropyl cellulose were loaded into a fluidized bed processor bowl for granulation.

4. The material of step 3 was granulated in fluidized bed processor by spraying the solution of step 2 on it.

5. The granules were dried, and then filled into hard gelatin capsules.

### Example 7

| S. No. | Ingredients | Quantity (% w/w) |
|---|---|---|
| 1. | Isotretinoin | 10.50 |
| 2. | Lactose anhydrous | 63.00 |
| 3. | Butylated hydroxy anisole | 0.25 |
| 4. | Povidone | 5.25 |
| 5. | Polyoxyl 40 Hydrogenated Castor Oil | 5.25 |
| 6. | Hydroxypropylmethyl cellulose | 10.50 |
| 7. | L-Hydroxypropyl cellulose | 5.25 |
| 8. | Dichloromethane | q.s. |
| 9. | Ethanol | q.s. |

### Procedure

1. Povidone, Polyoxyl 40 Hydrogenated Castor Oil and hydroxypropylmethyl cellulose were added to a mixture of dichloromethane and ethanol (in a ratio of 70:30) under stirring to form a clear solution.

2. Butylated hydroxy anisole and isotretinoin were dissolved in the solution of step 1.

3. Lactose and L-hydroxy propyl cellulose were loaded into a fluidized bed processor bowl for granulation.

4. The material of step 3 was granulated in fluidized bed processor by spraying the solution of step 2 on it.

5. The granules were dried and filled into hard gelatin capsules.

### Example 8

| S. No. | Ingredients | Quantity (% w/w) |
|---|---|---|
| 1. | Isotretinoin | 10.23 |
| 2. | Lactose anhydrous | 61.37 |
| 3. | Butylated hydroxy anisole | 0.25 |
| 4. | Povidone | 5.12 |
| 5. | Polyoxyl 40 Hydrogenated Castor Oil | 5.12 |
| 6. | Hydroxypropylmethyl cellulose | 12.79 |
| 7. | L-Hydroxypropyl cellulose | 5.12 |
| 8. | Dichloromethane | q.s. |
| 9. | Ethanol | q.s. |

### Procedure

1. Povidone and Polyoxyl 40 Hydrogenated Castor Oil were added to a mixture of dichloromethane and ethanol (in a ratio of 70:30) under stirring to form a clear solution.

2. Butylated hydroxy anisole and isotretinoin were dissolved in the solution of step 1.

3. Lactose and L-hydroxypropyl cellulose were loaded into a fluidized bed processor bowl for granulation.

4. The material of step 3 was granulated in fluidized bed processor by spraying the solution of step 2 on it.

5. The granules were dried, then mixed with hydroxypropylmethyl cellulose, and then filled into hard gelatin capsules.

### Example 9

| S. No. | Ingredients | Quantity (% w/w) |
|---|---|---|
| 1. | Isotretinoin | 11.89 |
| 2. | Lactose anhydrous | 71.32 |
| 3. | Butylated hydroxy anisole | 0.30 |
| 4. | Povidone | 5.94 |
| 5. | Stearoyl Macrogol-32 glycerides | 5.94 |
| 6. | Xanthan gum | 4.46 |
| 7. | Magnesium stearate | 0.15 |
| 8. | Dichloromethane | q.s. |
| 9. | Ethanol | q.s. |

### Procedure

1. Povidone, Stearoyl Macrogol-32 glycerides and xanthan gum were dissolved in a mixture of dichloromethane and ethanol (in a ratio of 70:30) to form a clear solution.

2. Butylated hydroxy anisole and isotretinoin were dissolved in the solution of step 1.

3. Lactose and L-hydroxy propyl cellulose were loaded into a fluidized bed processor bowl for granulation.

4. The material of step 3 was granulated in fluidized bed processor by spraying the solution of step 2 on it.

5. The granules were dried, and then mixed with extra-granular excipients to form a blend.

6. The blend of step 5 was compressed into tablets.

### Example 10

| S. No. | Ingredients | Quantity (% w/w) |
|---|---|---|
| 1. | Isotretinoin | 5.71 |
| 2. | Hydroxypropylmethyl cellulose | 2.87 |
| 3. | Meglumine | 0.71 |
| 4. | Butylated hydroxy toluene | 0.08 |
| 5. | Propyl gallate | 0.05 |
| 6. | Lactose | 27.14 |
| 7. | Mannitol | 27.43 |
| 8. | Polyethoxylated castor oil | 3.43 |
| 9. | Hydroxypropylmethyl cellulose | 21.43 |
| 10. | Magnesium stearate | 0.71 |
| 11. | Colloidal silicon dioxide | 4.86 |
| 12. | Hydrogenated castor oil | 5.58 |
| 13. | Purified water | q.s. |

### Procedure

1. Hydroxypropylmethyl cellulose and meglumine were dissolved in water.

2. Butylated hydroxy toluene and propyl gallate were dispersed in the solution of step 1.

3. Isotretinoin was suspended in the dispersion of step 2.

4. The drug suspension of step 3 was milled in a Dyno®-Mill containing zirconium beads to achieve a particle size of isotretinoin such that the D₉₀ was about 2 μm.

5. Polyethoxylated castor oil was added to the dispersion of step 4 at the end of the milling process.

6. Lactose and mannitol were loaded into a fluidized bed processor bowl for granulation.

7. The dispersion of step 5 was sprayed over the material of step 6 to form granules.

8. The granules obtained in step 7 were dried, and then mixed with hydroxypropylmethyl cellulose K4M, magnesium stearate, colloidal silicon dioxide, and Hydrogenated castor oil to form a blend.

9. The blend of step 8 was compressed into tablets.

### Example 11

| S. No. | Ingredients | Quantity (% w/w) |
|---|---|---|
| 1. | Isotretinoin | 5.71 |
| 2. | Hydroxypropylmethyl cellulose | 2.87 |
| 3. | Meglumine | 0.71 |
| 4. | Butylated hydroxy toluene | 0.08 |
| 5. | Propyl gallate | 0.05 |
| 6. | Lactose | 27.14 |
| 7. | Mannitol | 27.43 |
| 8. | Polyethoxylated castor oil | 3.43 |
| 9. | Hydroxypropylmethyl cellulose K4M | 14.29 |
| 10. | Hydroxypropylmethyl cellulose K100LV | 7.14 |
| 11. | Magnesium stearate | 0.71 |
| 12. | Colloidal silicon dioxide | 4.87 |
| 13. | Hydrogenated castor oil | 5.57 |
| 14. | Purified water | q.s. |

### Procedure

1. Hydroxypropylmethyl cellulose and meglumine were dissolved in water.

2. Butylated hydroxy toluene and propyl gallate were dispersed in the solution of step 1.

3. Isotretinoin was suspended in the dispersion of step 2.

4. The drug suspension of step 3 was milled in a Dyno®-Mill containing zirconium beads to achieve a particle size of isotretinoin such that the D₉₀ was about 2 μm.

5. Polyethoxylated castor oil was added to the dispersion of step 4 at the end of the milling process.

6. Lactose and mannitol were loaded into a fluidized bed processor bowl for granulation.

7. The dispersion of step 5 was sprayed over the material of step 6 to form granules.

8. The granules obtained in step 7 were dried, and then mixed with hydroxypropylmethyl cellulose K4M, hydroxypropylmethyl cellulose K100LV, magnesium stearate, colloidal silicon dioxide, and Hydrogenated castor oil to form a blend.

9. The blend of step 8 was compressed into tablets.

### Example 12

| S. No. | Ingredients | Quantity (% w/w) |
|---|---|---|
| 1. | Isotretinoin | 5.71 |
| 2. | Hydroxypropylmethyl cellulose | 2.87 |
| 3. | Meglumine | 0.71 |
| 4. | Butylated hydroxy toluene | 0.08 |
| 5. | Propyl gallate | 0.05 |
| 6. | Lactose | 27.14 |
| 7. | Mannitol | 27.43 |
| 8. | Polyethoxylated castor oil | 3.43 |
| 9. | Hydroxypropylmethyl cellulose K4M | 14.29 |
| 10. | Hydroxypropylmethyl cellulose K100M | 7.14 |
| 11. | Magnesium stearate | 0.71 |
| 12. | Colloidal silicon dioxide | 4.87 |
| 13. | Hydrogenated castor oil | 5.57 |
| 14. | Purified water | q.s. |

### Procedure

1. Hydroxypropylmethyl cellulose and meglumine were dissolved in water.

2. Butylated hydroxyl toluene and propyl gallate were dispersed in the solution of step 1.

3. Isotretinoin was suspended in the dispersion of step 2.

4. The drug suspension of step 3 was milled in a Dyno®-Mill containing zirconium beads to achieve a particle size of isotretinoin such that the D₉₀ was about 2 μm.

5. Polyethoxylated castor oil was added to the dispersion of step 4 at the end of the milling process.

6. Lactose and mannitol were loaded into a fluidized bed processor bowl for granulation.

7. The dispersion of step 5 was sprayed over the material of step 6 to form granules.

8. The granules obtained in step 7 were dried, and then mixed with hydroxypropylmethyl cellulose K4M, hydroxypropylmethyl cellulose K100LV, magnesium stearate, colloidal silicon dioxide, and Hydrogenated castor oil to form a blend.

9. The blend of step 8 was compressed into tablets.

### Example 13

| S. No. | Ingredients | Quantity (% w/w) |
|---|---|---|
| 1. | Isotretinoin | 4.00 |
| 2. | Hydroxypropylmethyl cellulose | 2.01 |
| 3. | Meglumine | 0.50 |
| 4. | Butylated hydroxy toluene | 0.05 |
| 5. | Propyl gallate | 0.04 |
| 6. | Lactose | 19.00 |
| 7. | Mannitol | 19.20 |
| 8. | Polyethoxylated castor oil | 2.40 |
| 9. | Crospovidone | 25.00 |
| 10. | Polyethylene oxide | 23.90 |
| 11. | Magnesium stearate | 0.50 |
| 12. | Colloidal silicon dioxide | 3.40 |
| 13. | Purified water | q.s. |

### Procedure

1. Hydroxypropylmethyl cellulose and meglumine were dissolved in water.

2. Butylated hydroxy toluene and propyl gallate were dispersed in the solution of step 1.

3. Isotretinoin was suspended in the dispersion of step 2.

4. The drug suspension of step 3 was milled in a Dyno®-Mill containing zirconium beads to achieve a particle size of isotretinoin such that the D₉₀ was about 2 μm.

5. Polyethoxylated castor oil was added to the dispersion of step 4 at the end of the milling process.

6. Lactose and mannitol were loaded into a fluidized bed processor bowl for granulation.

7. The dispersion of step 5 was sprayed over the material of step 6 to form granules.

8. The granules obtained in step 7 were dried, and then mixed with polyethylene oxide, crospovidone, magnesium stearate, and colloidal silicon dioxide to form a blend.

9. The blend of step 8 was compressed into tablets.

### Example 14

| S. No. | Ingredients | Quantity (% w/w) |
|---|---|---|
| 1. | Isotretinoin | 4.00 |
| 2. | Hydroxypropylmethyl cellulose | 2.00 |
| 3. | Meglumine | 0.50 |
| 4. | Butylated hydroxy toluene | 0.05 |
| 5. | Propyl gallate | 0.04 |
| 6. | Lactose | 19.00 |
| 7. | Mannitol | 19.21 |
| 8. | Polyethoxylated castor oil | 2.40 |
| 9. | Crospovidone | 25.00 |
| 10. | Hydroxypropylmethyl cellulose K4M | 20.00 |
| 11. | Hydroxypropylmethyl cellulose K100LV | 3.90 |
| 12. | Magnesium stearate | 0.50 |
| 13. | Colloidal silicon dioxide | 3.40 |
| 14. | Purified water | q.s. |

### Procedure

1. Hydroxypropylmethyl cellulose and meglumine were dissolved in water.

2. Butylated hydroxy toluene and propyl gallate were dispersed in the solution of step 1.

3. Isotretinoin was suspended in the dispersion of step 2.

4. The drug suspension of step 3 was milled in a Dyno®-Mill containing zirconium beads to achieve a particle size of isotretinoin such that the D₉₀ was about 2 μm.

5. Polyethoxylated castor oil was added to the dispersion of step 4 at the end of the milling process.

6. Lactose and mannitol were loaded into a fluidized bed processor bowl for granulation.

7. The dispersion of step 5 was sprayed over the material of step 6 to form granules.

8. The granules obtained in step 7 were dried, and then mixed with extra-granular hydroxypropylmethyl cellulose K4M, hydroxypropylmethyl cellulose K100LV CR, crospovidone, magnesium stearate, and colloidal silicon dioxide to form a blend.

9. The blend of step 8 was compressed into tablets.

### Example 15

| | | |
|---|---|---|
| S. No. | Ingredients | Quantity (% w/w) |

Core tablets
Intra-granular part

| | | |
|---|---|---|
| 1. | Isotretinoin | 5.71 |
| 2. | Hydroxypropylmethyl cellulose | 2.87 |
| 3. | Meglumine | 0.71 |
| 4. | Butylated hydroxy toluene | 0.08 |
| 5. | Propyl gallate | 0.05 |
| 6. | Lactose | 27.14 |
| 7. | Mannitol | 27.43 |
| 8. | Polyethoxylated castor oil | 3.43 |
| 9. | Purified water | q.s. |

Extra-granular part

| | | |
|---|---|---|
| 10. | Lactose | 21.43 |
| 11. | Magnesium stearate | 0.71 |
| 12. | Colloidal silicon dioxide | 4.87 |
| 13. | Hydrogenated castor oil | 5.57 |

Coating solution/dispersion

| | | |
|---|---|---|
| 14. | Ethyl cellulose | 60.60 |
| 15. | Hydroxypropylmethyl cellulose | 30.30 |
| 16. | Triethyl citrate | 9.10 |
| 17. | Isopropyl alcohol | q.s. |
| 18. | Purified water | q.s. |

### Procedure

### Core Tablets

1. Hydroxypropylmethyl cellulose and meglumine were dissolved in water.

2. Butylated hydroxy toluene and propyl gallate were dispersed in the solution of step 1.

3. Isotretinoin was suspended in the dispersion of step 2.

4. The drug suspension of step 3 was milled in a Dyno®-Mill containing zirconium beads to achieve a particle size of isotretinoin such that the D₉₀ was about 2 μm.

5. Polyethoxylated castor oil was added to the dispersion of step 4 at the end of the milling process.

6. Lactose and mannitol were loaded into a fluidized bed processor bowl for granulation.

7. The dispersion of step 5 was sprayed over the material of step 6 to form granules.

8. The granules obtained in step 7 were dried, and then mixed with extra-granular lactose, magnesium stearate, colloidal silicon dioxide, and Hydrogenated castor oil to form a blend.

9. The blend of step 8 was compressed into tablets.

### Coating

10. Ethyl cellulose was dissolved in a mixture of isopropyl alcohol and water in a ratio of 80:20 to make a 6% w/w solution.

11. Hydroxypropylmethyl cellulose and triethyl citrate were added to the solution of step 10.

12. The core tablets were coated with the coating solution of step 11.

### Example 16

| | | |
|---|---|---|
| S. No. | Ingredients | Quantity (% w/w) |

Core
Intra-granular part

| | | |
|---|---|---|
| 1 | Isotretinoin | 5.71 |
| 2 | Hydroxypropylmethyl cellulose | 2.86 |
| 3 | Meglumine | 0.71 |
| 4 | Butylated hydroxy toluene | 0.08 |
| 5 | Propyl gallate | 0.05 |
| 6 | Lactose | 27.14 |
| 7 | Mannitol | 27.43 |
| 8 | Polyethoxylated castor oil | 3.44 |
| 9 | Water | q.s. |

Extra-granular part

| | | |
|---|---|---|
| 10 | Lactose | 21.43 |
| 11 | Magnesium stearate | 0.71 |
| 12 | Colloidal silicon dioxide | 4.87 |
| 13 | Hydrogenated castor oil | 5.57 |

Coating solution/dispersion

| | | |
|---|---|---|
| 14 | Ethyl cellulose | 64.48 |
| 15 | Hydroxypropylmethyl cellulose | 25.84 |
| 16 | Triethyl citrate | 9.68 |
| 17 | Isopropyl alcohol | q.s. |
| 18 | Purified water | q.s. |

### Procedure

### Core Tablets

1. Hydroxypropylmethyl cellulose and meglumine were dissolved in water.

2. Butylated hydroxy toluene and propyl gallate were dispersed in the solution of step 1.

3. Isotretinoin was suspended in the dispersion of step 2.

4. The drug suspension of step 3 was milled in a Dyno®-Mill containing zirconium beads to achieve a particle size of isotretinoin such that the D₉₀ was about 2 μm.

5. Polyethoxylated castor oil was added to the dispersion of step 4 at the end of the milling process.

6. Lactose and mannitol were loaded into a fluidized bed processor bowl for granulation.

7. The dispersion of step 5 was sprayed over the material of step 6 to form granules.

8. The granules obtained in step 7 were dried, and then mixed with extra-granular lactose, magnesium stearate, colloidal silicon dioxide, and Hydrogenated castor oil to form a blend.

9. The blend of step 8 was compressed into tablets.

### Coating

10. Ethyl cellulose was dissolved in a mixture of isopropyl alcohol and water in a ratio of 80:20 to make a 6% w/w solution.

11. Hydroxypropylmethyl cellulose and triethyl citrate were added to the solution of step 10.

12. The core tablets were coated with coating solution of step 11.

### Example 17

| S. No. | Ingredients | Quantity (% w/w) |
|---|---|---|
| 1. | Isotretinoin | 2.41 |
| 2. | Propylene Glycol Monocaprylate | 17.24 |
| 3. | Transcutol | 34.49 |
| 4. | Polyethoxylated castor oil | 17.24 |
| 5. | Butylated hydroxy toluene | 0.04 |
| 6. | Propyl gallate | 0.03 |
| 7. | Neusilin ® | 28.55 |

### Procedure:

1. Propylene Glycol Monocaprylate, Transcutol, and Polyethoxylated castor oil were mixed to form a solution under stirring.

2. Butylated hydroxy toluene and propyl gallate were dissolved in the solution of step 1.

3. Isotretinoin was dissolved in the solution of step 2 with stirring.

4. The solution of step 3 was adsorbed onto Neusilin® to form a powder.

5. The powder obtained in step 5 was filled into capsules.

### Example 18

| S. No. | Ingredients | Quantity (% w/w) |
|---|---|---|
| 1. | Isotretinoin | 10.26 |
| 2. | Hydroxypropylmethyl cellulose | 2.56 |
| 3. | Meglumine | 2.56 |
| 4. | Poloxamer | 1.28 |
| 5. | Butylated hydroxy anisole | 0.13 |
| 6. | Propyl gallate | 0.13 |
| 7. | Dichloromethane | q.s. |
| 8. | Ethanol absolute | q.s. |

Compression - Part A

| | | |
|---|---|---|
| 9. | Anhydrous lactose | 33.71 |
| 10. | Colloidal silicon dioxide | 0.23 |
| 11. | Sodium stearyl fumarate | 0.29 |

Compression - Part B

| | | |
|---|---|---|
| 12. | Hydroxypropylmethyl cellulose | 46.16 |
| 13. | Anhydrous lactose | 1.13 |
| 14. | Colloidal silicon dioxide | 0.69 |
| 15. | Sodium stearyl fumarate | 0.87 |

### Procedure:

1. Isotretinoin, hydroxypropylmethyl cellulose, meglumine, poloxamer, butylated hydroxy anisole, and propyl gallate were added to a mixture of dichloromethane and ethanol under stirring to form a clear solution.

2. The solution of step 1 was processed into dry form.

### Compression—Part A

3. The dried blend (25%) of step 2 was mixed with anhydrous lactose, colloidal silicon dioxide, and sodium stearyl fumarate to form a blend.

4. The blend of step 3 was compressed into tablets.

### Compression—Part B

5. The dried blend (75%) of step 2 was mixed with anhydrous lactose, hydroxypropyl methylcellulose, colloidal silicon dioxide, and sodium stearyl fumarate to form a blend.

6. The blend of step 5 was compressed into tablets.

### Filling of Capsules

7. One tablet each of part A and part B were filled into a hard gelatin capsule.

### Example 19

| S. No. | Ingredients | Quantity (% w/w) |
|---|---|---|
| 1 | Isotretinoin | 11.11 |
| 2 | Hydroxypropylmethyl cellulose | 2.78 |
| 3 | Meglumine | 2.78 |
| 4 | Poloxamer | 1.39 |
| 5 | Butylated hydroxy anisole | 0.14 |
| 6 | Propyl gallate | 0.14 |
| 7 | Dichloromethane | q.s. |
| 8 | Ethanol absolute | q.s. |

Compression

| | | |
|---|---|---|
| 9 | Hydroxypropylmethyl cellulose | 66.66 |
| 10 | Xanthan gum | 1.25 |
| 11 | Sodium alginate | 0.42 |
| 12 | Sodium bicarbonate | 8.33 |
| 13 | Anhydrous lactose | 2.75 |
| 14 | Colloidal silicon dioxide | 1.00 |
| 15 | Sodium stearyl fumarate | 1.25 |

### Procedure:

1. Isotretinoin, hydroxypropyl methylcellulose, meglumine, poloxamer, butylated hydroxy anisole, and propyl gallate were added in a mixture of dichloromethane and ethanol under stirring to form a clear solution.

2. The solution of step 1 was processed into dry form.

3. The dried blend of step 2 was mixed with anhydrous lactose, hydroxypropyl methylcellulose, xanthan gum, sodium alginate, sodium bicarbonate, colloidal silicon dioxide, and sodium stearyl fumarate to form a blend.

4. The blend of step 3 was compressed into tablets.

5. One tablet was filled into a hard gelatin capsule.

### Example 20

| S. No. | Ingredients | Quantity (% w/w) |
|---|---|---|
| 1 | Isotretinoin | 9.78 |
| 2 | Hydroxypropylmethyl cellulose | 2.44 |
| 3 | Meglumine | 2.44 |
| 4 | Poloxamer | 1.22 |
| 5 | Butylated hydroxy anisole | 0.12 |
| 6 | Propyl gallate | 0.12 |
| 7 | Dichloromethane | q.s. |
| 8 | Ethanol absolute | q.s. |

Compression - Part A

| | | |
|---|---|---|
| 9 | Anhydrous lactose | 32.13 |
| 10 | Colloidal silicon dioxide | 0.22 |
| 11 | Sodium stearyl fumarate | 0.27 |

Compression - Part B

| | | |
|---|---|---|
| 12 | Hydroxypropylmethyl cellulose | 43.99 |
| 13 | Anhydrous lactose | 1.08 |
| 14 | Colloidal silicon dioxide | 0.66 |
| 15 | Sodium stearyl fumarate | 0.83 |

Coating - Part B

| | | |
|---|---|---|
| 16 | Eudragit | 3.62 |
| 17 | Triethyl citrate | 0.36 |
| 18 | Talc | 0.72 |
| 19 | Isopropyl alcohol | q.s. |
| 20 | Acetone | q.s. |

### Procedure:

1. Isotretinoin, hydroxypropylmethyl cellulose, meglumine, poloxamer, butylated hydroxy anisole, and propyl gallate were added to a mixture of dichloromethane and ethanol (in a ratio of 70:30) under stirring to form a clear solution.

2. The solution of step 1 was processed into dry form.

### Compression—Part A

3. The dried blend (25%) of step 2 was mixed with anhydrous lactose, colloidal silicon dioxide, and sodium stearyl fumarate to form a blend.

4. The blend of step 3 was compressed into tablets.

### Compression—Part B

5. The dried blend (75%) of step 2 was mixed with anhydrous lactose, hydroxypropyl methylcellulose, colloidal silicon dioxide, and sodium stearyl fumarate to form a blend.

6. The blend of step 1 was compressed into tablets.

### Coating—Part B

7. Isopropyl alcohol and acetone were mixed under stirring.

8. Eudragit was dissolved into half of the solvent mixture of step 7 under stirring to obtain a clear solution.

9. Talc was dispersed into the other half of the solvent mixture of step 7 under stirring.

10. The dispersion of step 9 was added into solution of step 8 under stirring, followed by the addition of triethyl citrate.

11. The dispersion of step 10 was stirred, and then the core tablets of Part B were coated using the dispersion.

### Filling of Capsules

12. One tablet each of part A and part B were filled into a hard gelatin capsule.

### Example 21

| | | Example | Example |
|---|---|---|---|
| | | 21 (A) | 21 (B) |
| S. No. | Ingredients | Quantity (% w/w) | |
| 1 | Isotretinoin | 8.62 | 8.62 |
| 2 | Butylated hydroxy | 0.09 | 0.09 |
| | anisole | | |
| 3 | Oleic acid | 46.98 | 46.98 |
| 4 | Polysorbate 80 | 2.16 | 2.16 |
| 5 | Ethyl cellulose | 7.67 | 7.67 |
| 6 | Soybean oil | 34.48 | 34.48 |
| 7 | Gelatin | q.s. | q.s. |
| 8 | Purified water | q.s. | q.s. |
| 9 | FDA approved color | q.s. | q.s. |

| | |
|---|---|
| Coating ingredients | Coat composition (mg/capsule) |

Seal coating (3% w/w)

| | | | |
|---|---|---|---|
| 10 | Hydroxypropyl | 9.00 | 9.00 |
| | methylcellulose | | |
| 11 | Isopropyl alcohol | q.s. | q. s. |
| 12 | Dichloromethane | q.s. | q. s. |

Functional Coating

| | | 7% w/w | 9% w/w |
|---|---|---|---|
| 13 | Ethyl cellulose | 8.83 | 11.35 |
| 14 | Hydroxypropylmethyl | 10.59 | 13.62 |
| | cellulose | | |
| 15 | Triethyl citrate | 2.21 | 2.84 |
| 16 | Isopropyl alcohol | q.s. | q.s. |
| 17 | Dichloromethane | q.s. | q.s. |

### Procedure

1. Butylated hydroxy anisole and polysorbate 80 were dissolved in soyabean oil to form a clear solution.

2. Isotretinoin was added to the solution of step 1 under stirring to obtain a uniform suspension.

3. The suspension of step 2 was milled to get a particle size of isotretinoin such that the D₉₀ was about 19 μm.

4. The suspension of step 3 was filled into hard gelatin capsules.

5. Gelatin was soaked in purified water at 60° C. to 90° C. along with color to get a uniform solution.

6. The filled capsules of step 4 were band-sealed using the gelatin solution of step 5.

7. Hydroxypropylmethyl cellulose was dissolved in dichloromethane, and then isopropyl alcohol was added into the mixture to form a clear solution.

8. The band-sealed capsules of step 6 were seal-coated using the solution of step 7.

9. Ethyl cellulose was dissolved in isopropyl alcohol.

10. Hydroxypropylmethyl cellulose was dissolved in dichloromethane, and then isopropyl alcohol was added to the mixture to obtain a clear solution.

11. The solutions of step 9 and step 10 were mixed under stirring while adding triethyl citrate.

12. The seal-coated capsules of step 8 were coated with the solution of step 11.

### Example 22

| S. No. | Ingredients | Quantity (w/w) |
|---|---|---|
| 1 | Isotretinoin | 12.35 |
| 2 | Butylated hydroxy anisole | 0.12 |
| 3 | Oleic acid | 49.39 |
| 4 | Soybean oil | 21.15 |
| 5 | Ammonio methacrylate copolymer | 15.73 |
| 6 | Triethyl citrate | 1.26 |

### Procedure

1. Butylated hydroxy anisole was dissolved in oleic acid to form a clear solution.

2. Isotretinoin was added to the solution of step 1 under stirring to obtain a uniform suspension.

3. The suspension of step 2 was milled to get a particle size of isotretinoin such that the D₉₀ was about 7 μm.

4. Ammonio methacrylate copolymer and triethyl citrate were dissolved in soybean oil under stirring, to obtain a uniform suspension.

5. The suspension of step 4 was added into the suspension of step 3 under stirring to obtain a uniform suspension.

6. The suspension of step 5 was filled into hard gelatin capsules.

### Example 23

| S. No. | Ingredients | Quantity (% w/w) |
|---|---|---|
| 1 | Isotretinoin | 11.11 |
| 2 | Butylated hydroxy anisole | 0.27 |
| 3 | Oleic acid | 44.44 |
| 4 | Polysorbate 80 | 1.39 |
| 5 | Ethyl cellulose | 5.65 |
| 6 | Diethylene glycol monoethyl ether | 35.85 |
| 7 | Triethyl citrate | 1.29 |

### Procedure

1. Butylated hydroxy anisole and polysorbate 80 were dissolved in oleic acid to form a clear solution.

2. Isotretinoin was added to the solution of step 1 under stirring to obtain a uniform suspension.

3. The suspension of step 2 was milled to get a particle size of isotretinoin such that the D₉₀ was about 26 μm.

4. Ethyl cellulose and triethyl citrate were added to diethylene glycol monoethyl ether under stirring to form a solution.

5. The solution of step 4 was added into the suspension of step 3, under stirring, to get a uniform suspension.

6. The suspension of step 5 was filled into hard gelatin capsules.

### Example 24

| | | |
|---|---|---|
| S. No. | Ingredients | Quantity (% w/w) |

Dyno-milled dispersion

| | | |
|---|---|---|
| 1 | Isotretinoin | 4.46 |
| 2 | Hydroxypropylmethyl cellulose | 2.23 |
| 3 | Meglumine | 0.55 |
| 4 | Butylated hydroxy toluene | 0.06 |
| 5 | Propyl gallate | 0.04 |
| 6 | Water | q.s. |

Granulation

| | | |
|---|---|---|
| 7 | Mannitol | 16.74 |
| 8 | Polyethoxylated castor oil | 0.11 |

Drug layer

| | | |
|---|---|---|
| 9 | Polyethylene Oxide | 33.50 |
| 10 | Sodium Chloride | 6.79 |
| 11 | Magnesium Stearate | 0.45 |

Push layer

| | | |
|---|---|---|
| 12 | Polyethylene Oxide | 17.30 |
| 13 | Sodium Chloride | 6.70 |
| 14 | Magnesium Stearate | 0.21 |
| 15 | Ferric oxide red | 0.21 |

Seal coat

| | | |
|---|---|---|
| 16 | Hydroxypropylmethyl cellulose | 2.41 |
| 17 | Polyethylene glycol | 0.27 |
| 18 | Water | q.s. |

Functional coat

| | | |
|---|---|---|
| 19 | Cellulose acetate | 3.90 |
| 20 | Polyethylene glycol | 1.16 |
| 21 | Acetone | q.s. |
| 22 | Water | q.s. |

Film-coating

| | | |
|---|---|---|
| 23 | Opadry ® Brown 03F565089 | 2.91 |
| 24 | Water | q.s. |

### Procedure:

### Dyno-Milled Dispersion

1. Hydroxypropylmethyl cellulose and meglumine were dissolved in water.

2. Butylated hydroxyl toluene and propyl gallate were dispersed in the solution of step 1.

3. Isotretinoin was added to the dispersion of step 2.

4. The drug dispersion of step 3 was milled in a Dyno®-Mill containing zirconium beads to achieve a particle size of isotretinoin such that the D₉₀ was about 2 μm.

### Granulation

5. Mannitol was sifted through a sieve and loaded into a fluidized bed granulator.

6. Polyethoxylated castor oil was added to the Dyno-milled dispersion of step 4 under stirring.

7. The dispersion of step 6 was sprayed over the loaded mannitol of step 5 to form granules.

### Drug Layer

8. The granules obtained in step 7 were mixed with previously sifted polyethylene oxide, sodium chloride, and magnesium stearate to form a drug blend.

### Polymer Layer

9. Polyethylene oxide, sodium chloride and magnesium stearate were sifted through a sieve and blended together to form a push layer blend.

### Compression

10. The blends of the drug layer and the push layer were loaded into respective hoppers of a bilayer compression machine and compressed into bilayered tablets.

### Seal Coating

11. Hydroxypropylmethyl cellulose and polyethylene glycol were dissolved in water to form a coating solution.

12. The tablets of step 10 were coated with the solution of step 11.

### Functional Coating

13. Cellulose acetate was dispersed in acetone to form a clear solution.

14. Polyethylene glycol was dissolved in purified water to form a clear solution.

15. The solutions of step 13 and step 14 were mixed together to form a clear solution.

16. The seal coated tablets of step 12 were coated with the solution of step 15.

### Film-Coating

17. The tablets were film-coated with a 10% dispersion of Opadry® Brown 03F565089 in purified water.

### Example 25

| | | |
|---|---|---|
| S. No. | Ingredients | Quantity (% w/w) |

Dyno-milled dispersion

| | | |
|---|---|---|
| 1. | Isotretinoin | 3.88 |
| 2. | Hydroxypropylmethyl cellulose | 1.95 |
| 3. | Meglumine | 0.49 |
| 4. | Butylhydroxy toluene | 0.05 |
| 5. | Propyl gallate | 0.04 |
| 6. | Water | q.s. |

Granulation

| | | |
|---|---|---|
| 7. | Mannitol | 18.64 |
| 8. | Anhydrous lactose | 18.45 |
| 9. | Polyethoxylated castor oil | 0.10 |

Compression

| | | |
|---|---|---|
| 10. | Anhydrous lactose | 5.05 |
| 11. | Crospovidone | 28.16 |
| 12. | Magnesium stearate | 0.49 |
| 13. | Colloidal silicon dioxide | 0.39 |
| 14. | Polyethylene oxide WSR 303 | 11.64 |
| 15. | Polyethylene oxide N80 | 7.76 |

Film-coating

| | | |
|---|---|---|
| 16. | Opadry ® Brown 03F565089 | 2.91 |
| 17. | Water | q.s. |

### Procedure:

### Dyno-Milled Dispersion

1. Hydroxypropylmethyl cellulose and meglumine were dissolved in water.

2. Butylated hydroxyl toluene and propyl gallate were dispersed in the solution of step 1.

3. Isotretinoin was added to the dispersion of step 2.

4. The drug dispersion of step 3 was milled in a Dyno®-Mill containing zirconium beads to achieve the particle size of isotretinoin such that D₉₀ was about 2μm.

### Granulation

5. Mannitol and lactose were sifted through a sieve and loaded into a fluidized bed granulator.

6. Polyethoxylated castor oil was added to the Dyno-milled dispersion of step 4 under stirring.

7. The dispersion of step 6 was sprayed over loaded material of step 5 to form granules.

### Compression

8. Granules obtained in step 7 were mixed with previously sifted anhydrous lactose, crospovidone, polyethylene oxide N80, polyethylene oxide WSR 303, sodium chloride, magnesium stearate and colloidal silicon dioxide to form the drug blend.

9. The blend of step 8 was compressed into tablets.

### Film-Coating

10. The tablets were film-coated with 10% dispersion of Opadryx® Brown 03F565089 in purified water.

### Example 26

| | | |
|---|---|---|
| S. No. | Ingredients | Quantity (% w/w) |

Dyno-milled dispersion

| | | |
|---|---|---|
| 1 | Isotretinoin | 3.74 |
| 2 | Hydroxypropylmethyl cellulose | 1.88 |
| 3 | Meglumine | 0.47 |
| 4 | Butylhydroxy toluene | 0.05 |
| 5 | Propyl gallate | 0.03 |
| 6 | Water | q.s. |

Granulation

| | | |
|---|---|---|
| 7 | Mannitol | 17.95 |
| 8 | Anhydrous lactose | 17.77 |
| 9 | Polyethoxylated castor oil | 0.09 |

MR layer blend

| | | |
|---|---|---|
| 10 | Crospovidone PPXL | 27.13 |
| 11 | Magnesium stearate | 0.4 |
| 12 | Colloidal silicon dioxide | 0.37 |
| 13 | Polyethylene oxide WSR 303 | 11.22 |
| 14 | Polyethylene oxide N80 | 7.48 |

IR layer blend

| | | |
|---|---|---|
| 15 | Anhydrous lactose | 7.01 |
| 16 | Crospovidone | 0.85 |
| 17 | Magnesium stearate | 0.19 |
| 18 | Colloidal silicon dioxide | 0.09 |
| 19 | Ferric oxide red | 0.07 |

Film-coating

| | | |
|---|---|---|
| 20 | Opadry ® Brown 03F565089 | 3.21 |
| 21 | Water | q.s. |

### Procedure:

### Dyno-Milled Dispersion

1. Hydroxypropylmethyl cellulose and meglumine were dissolved in water.

2. Butylated hydroxyl toluene and propyl gallate were dispersed in the solution of step 1.

3. Isotretinoin was added to the dispersion of step 2.

4. The drug dispersion of step 3 was milled in a Dyno®-Mill containing zirconium beads to achieve a particle size of isotretinoin such that the D₉₀ was about 2 μm.

### Granulation

5. Mannitol and lactose were sifted through a sieve and loaded into a fluidized bed granulator.

6. Polyethoxylated castor oil was added to the Dyno-milled dispersion of step 4 under stirring.

7. The dispersion of step 6 was sprayed over the loaded material of step 5 to form granules.

### MR Layer Blend

8. 75% of the granules obtained in step 7 were mixed with previously sifted crospovidone PPXL, polyethylene oxide N80, polyethylene oxide WSR 303, magnesium stearate, and colloidal silicon dioxide to form the MR layer blend.

### IR Layer Blend

9. 25% of the granules obtained in step 7 were mixed with previously sifted anhydrous lactose, crospovidone PPXL, ferric oxide red, magnesium stearate, and colloidal silicon dioxide to form the IR layer blend.

### Compression

10. The blends of the MR drug layer and the IR drug layer were loaded into respective hoppers of bilayer compression machines, and compressed into bi-layered tablets.

### Film-Coating

11. The tablets were film-coated with a 10% dispersion of Opadryx® Brown 03F565089 in purified water.

### Example 27

| S. No. | Ingredients | Quantity (mg/capsule) |
|---|---|---|
| Spray Drying | | 20 mg strength |
| 1 | Isotretinoin | 20.000 |
| 2 | Hydroxy propyl methyl cellulose | 5.000 |
| 3 | Meglumine | 5.000 |
| 4 | Poloxamer 188 | 5.000 |
| 5 | Polyethoxylated castor oil | 4.000 |
| 6 | Stearoyl Macrogol-32 glycerides | 6.000 |
| 7 | Stearic acid | 8.500 |
| 8 | Butylated hydroxy anisole | 0.250 |
| 9 | Propyl gallate | 0.250 |
| 10 | Dichloromethane | q.s. |
| 11 | Ethanol Absolute | q.s. |
| Part A | Immediate-release (IR) part | 10 mg strength |
| 1 | Spray dried blend | 27.000 |
| 2 | Anhydrous lactose | 161.750 |
| 3 | Fumed silica | 5.000 |
| 4 | Sodium stearyl fumarate | 6.250 |
| Part B | Extended-release (ER) tablet | 10 mg strength |
| 1 | Spray dried blend | 27.000 |
| 2 | Hydroxy propyl methyl cellulose | 275.000 |
| 3 | Anhydrous lactose | 96.750 |
| 4 | Sodium alginate | 3.750 |
| 5 | Sodium bicarbonate | 75.000 |
| 6 | Xanthan gum | 11.250 |
| 7 | Fumed silica | 5.000 |
| 8 | Sodium stearyl fumarate | 6.250 |
| Capsule Filling | | |
| 1 | Capsule shell | Part A + Part B |

### Procedure:

### Spray Drying

1. Dichloromethane and Ethanol were added in a stainless steel vessel.

2. Hydroxy propyl methyl cellulose, Meglumine, Poloxamer 188, butylated hydroxy anisole, propyl gallate, stearic acid, Polyethoxylated castor oil and Stearoyl Macrogol-32 glyceride were added into the solvent mixture of step 1.

3. Isotretinoin was added into step 2 under stirring to get a clear solution.

4. The solution of step 3 was spray dried to obtain the solid dispersion of isotretinoin.

### Part A—Immediate-Release (IR) Tablet

5. Spray dried blend of step 4 and anhydrous lactose were sifted through a suitable sieve.

6. Fumed silica was sifted through a suitable sieve and mixed with the material of step 5.

7. Sodium stearyl fumarate was sifted through a suitable sieve and mixed with the blend of step 6.

8. The blend of step 7 was compressed in suitable size tablet.

### Part B—Extended-Release (ER) Tablet

9. Remaining part of spray dried blend of step 4, xanthan gum, sodium alginate, sodium bicarbonate, hydroxyl propyl cellulose and anhydrous lactose were sifted through a suitable sieve.

10. Fumed silica was sifted through a suitable sieve and mixed with the blend of step 9.

11. Sodium stearyl fumarate was sifted through a suitable sieve and mixed with the blend of step 10.

12. The blend of step 11 was compressed into suitable size tablet.

### Capsule Filling

13. Part A and Part B of isotretinoin tablets were filled in suitable size capsule shell.

### Example 28

| S. No. | Ingredients | Quantity (mg/capsule) |
|---|---|---|
| Spray Drying | | 20 mg strength |
| 1 | Isotretinoin | 20.000 |
| 2 | Hydroxy propyl methyl cellulose | 5.000 |
| 3 | Meglumine | 5.000 |
| 4 | Poloxamer 188 | 5.000 |
| 5 | Polyethoxylated castor oil | 4.000 |
| 6 | Stearoyl Macrogol-32 glycerides | 6.000 |
| 7 | Stearic acid | 8.500 |
| 8 | Butylated hydroxy anisole | 0.250 |
| 9 | Propyl gallate | 0.250 |
| 10 | Dichloromethane | q.s. |
| 11 | Ethanol Absolute | q.s. |
| Part A | Immediate-release (IR) part | 10 mg strength |
| 1 | Spray dried blend | 27.000 |
| 2 | Anhydrous lactose | 161.750 |
| 3 | Fumed silica | 5.000 |
| 4 | Sodium stearyl fumarate | 6.250 |
| Part B | Extended-release (ER) part | 10 mg strength |
| 1 | Spray dried blend | 27.000 |
| 2 | Polyethylene oxide | 105.000 |
| 3 | Anhydrous lactose | 356.750 |
| 4 | Fumed silica | 5.000 |
| 5 | Sodium stearyl fumarate | 6.250 |
| Capsule Filling | | |
| 1 | Capsule shell | Part A + Part B |

### Procedure:

### Spray Drying

1. Dichloromethane and Ethanol were added in a stainless steel vessel.

2. Hydroxy propyl methyl cellulose, meglumine, poloxamer 188, butylated hydroxy anisole, propyl gallate, stearic acid, Polyethoxylated castor oil and Stearoyl Macrogol-32 glyceride were added into the solvent mixture of step 1 under stirring.

3. Isotretinoin was added into step 2 under stirring to get a clear solution.

4. The solution of step 3 was spray dried to obtain the solid dispersion of isotretinoin.

### Part A—Immediate-Release (IR) Tablet

5. Spray dried blend of step 4 and anhydrous lactose were sifted through a suitable sieve.

6. Fumed silica was sifted through a suitable sieve and mixed with the blend of step 5.

7. Sodium stearyl fumarate was sifted through a suitable sieve and mixed with the blend of step 6.

8. The blend of step 7 was compressed into suitable size tablet.

### Part B—Extended-Release (ER) Tablet

9. Remaining part of spray dried blend of step 4, polyethylene oxide and anhydrous lactose were sifted through a suitable sieve.

10. Fumed silica was sifted through a suitable sieve and mixed with the blend of step 9.

11. Sodium stearyl fumarate was sifted through a suitable sieve and mixed with the blend of step 10.

12. The blend of step 11 was compressed into suitable size tablet.

### Capsule Filling

13. Part A and Part B of isotretinoin tablets were filled into a suitable size capsule shell.

### Example 29

| S. No. | Ingredients | Quantity (mg/capsule) |
|---|---|---|
| Spray Drying | | 20 mg strength |
| 1 | Isotretinoin | 20.000 |
| 2 | Hydroxy methyl cellulose | 5.000 |
| 3 | Meglumine | 5.000 |
| 4 | Poloxamer 188 | 5.000 |
| 5 | Polyethoxylated castor oil | 4.000 |
| 6 | Stearoyl Macrogol-32 glyceride | 6.000 |
| 7 | Stearic acid | 8.500 |
| 8 | Butylated hydroxy anisole | 0.250 |
| 9 | Propyl gallate | 0.250 |
| 10 | Dichloromethane | q.s. |
| 11 | Ethanol Absolute | q.s. |
| Part A | Immediate-release (IR) part | 8 mg strength |
| 1 | Spray dried blend | 21.600 |
| 2 | Anhydrous lactose | 129.400 |
| 3 | Fumed silica | 4.000 |
| 4 | Sodium stearyl fumarate | 5.000 |
| Part B | Extended-release (ER) part | 8 mg strength |
| 1 | Spray dried blend | 21.600 |
| 2 | Hydroxy methyl cellulose | 90.000 |
| 3 | Anhydrous lactose | 15.850 |
| 4 | Fumed silica | 1.160 |
| 5 | Sodium stearyl fumarate | 1.390 |
| Part C | Immediate-release (IR) coated | 4 mg strength |
| 1 | Spray dried blend | 10.800 |
| 2 | Anhydrous lactose | 140.200 |
| 3 | Fumed silica | 4.000 |
| 4 | Sodium stearyl fumarate | 5.000 |
| | Delayed-release (DR) coating | |
| 5 | Eudragit L100 | 9.847 |
| 6 | Triethyl citrate | 0.984 |
| 7 | Talc | 1.969 |
| 8 | Isopropyl alcohol | q.s. |
| 9 | Acetone | q.s. |
| Capsule Filling | | |
| 1 | Capsule shell | Part A + Part B + Part C |

### Procedure

### Spray Drying

1. Dichloromethane and Ethanol were added in a stainless steel vessel.

2. Hydroxy propyl methyl cellulose, meglumine, poloxamer 188, butylated hydroxy anisole, propyl gallate, stearic acid, polyethoxylated castor oil and Stearoyl Macrogol-32 glyceride were added into the solvent mixture of step 1 under stirring.

3. Isotretinoin was added into step 2 under stirring to get a clear solution.

4. The solution of step 3 was spray dried to obtain solid dispersion of isotretinoin.

### Part A—Immediate-Release (IR) Tablet

5. Spray dried blend of step 4 and anhydrous lactose were sifted through a suitable sieve.

6. Fumed silica was sifted through a suitable sieve and mixed with the blend of step 5.

7. Sodium stearyl fumarate was sifted through a suitable sieve and mixed with the blend of step 6.

8. The blend of step 7 was compressed into suitable size tablet.

### Part B—Extended-Release (ER) Tablet

9. Spray dried blend of step 4, hydroxyl propyl methyl cellulose and anhydrous lactose were sifted through a suitable sieve.

10. Fumed silica was sifted through a suitable sieve and mixed with the blend of step 9.

11. Sodium stearyl fumarate was sifted through a suitable sieve and mixed with the blend of step 10.

12. The blend of step 11 was compressed into suitable size tablet.

### Part C—Delayed-Release (DR) Tablet

### Part-I

13. Remaining part of spray dried blend, anhydrous lactose were sifted through a suitable sieve.

14. Fumed silica was sifted through a suitable sieve and mixed with the blend of step 13.

15. Sodium stearyl fumarate was sifted through a suitable sieve and mixed with the blend of step 14.

16. The blend of step 15 was compressed in a suitable size tablet.

### Part-II

17. Isopropyl alcohol and acetone were mixed and Eudragit L100 was added into the solvent mixture under slow stirring.

18. Triethyl citrate followed by talc was added into the solvent mixture of step 17.

19. The compressed tablets of step 16 were coated with the solution of step 18 to obtain delayed release tablets.

### Capsule Filling

20. Part A, Part B and Part C of isotretinoin tablets were filled into suitable size capsule.

### Example 30

| S. No. | Ingredients | Quantity (mg/capsule) |
|---|---|---|
| Spray Drying | | 20 mg strength |
| 1 | Isotretinoin | 20.000 |
| 2 | Hydroxy methyl cellulose | 5.000 |
| 3 | Meglumine | 5.000 |
| 4 | Poloxamer 188 | 5.000 |
| 5 | Polyethoxylated castor oil | 4.000 |
| 6 | Stearoyl Macrogol-32 glycerides | 6.000 |
| 7 | Stearic acid | 8.500 |
| 8 | Butylated hydroxy anisole | 0.250 |
| 9 | Propyl gallate | 0.250 |
| 10 | Dichloromethane | q.s. |
| 11 | Ethanol Absolute | q.s. |
| Part A | Immediate-release (IR) part | 8 mg strength |
| 1 | Isotretinoin | 8.000 |
| 2 | Butylated hydroxy anisole | 0.045 |
| 3 | Polysorbate 80 | 2.220 |
| 4 | Refined Soyabean oil | 109.735 |
| Part B | Extended-release (ER) part | 12 mg strength |
| 1 | Spray dried blend | 32.400 |
| 2 | Hydroxy methyl cellulose | 90.000 |
| 3 | Anhydrous lactose | 5.050 |
| 4 | Fumed silica | 1.160 |
| 5 | Sodium stearyl fumarate | 1.390 |
| Capsule Filling | | |
| 1 | Capsule shell | Part A + Part B |
| 2 | Gelatin | q.s. |
| 3 | Purified water | q.s. |

### Procedure

### Spray Drying

1. Dichloromethane and Ethanol were added in a stainless steel vessel.

2. Hydroxy methyl cellulose, meglumine, poloxamer, butylated hydroxy anisole, propyl gallate, stearic acid, Polyethoxylated castor oil and Stearoyl Macrogol-32 glyceride were added into the solvent mixture of step 1 under stirring.

3. Isotretinoin was added into step 2 under stirring to get a clear solution.

4. The solution of step 3 was spray dried using spray dryer equipment.

### Part A—Immediate-Release (IR) Blend

5. Soyabean oil was weighed and taken in a suitable stainless steel vessel.

6. Butylated hydroxy anisole was added into the vessel of step 5 under stirring to get a clear solution.

7. Polysorbate 80 was added into the solution of step 6.

8. Isotretinoin was added into the solution of step 7 under stirring to get a uniform suspension.

9. The suspension of step 8 was mill dispersed using a Dyno-mill.

### Part B—Extended-Release (ER) Tablet

10. Spray dried blend of step 4, hydroxy propyl methyl cellulose and anhydrous lactose were sifted through a suitable sieve.

11. Fumed silica was sifted through a suitable sieve and mixed with the blend of step 11.

12. Sodium stearyl fumarate) was sifted through a suitable sieve and mixed with the blend of step 12.

13. The blend of step 13 was compressed into suitable size tablet.

### Capsule Filling

14. Isotretinoin dispersion of step 9 and Part B tablet obtained in step 13 was filled into specified capsule shell.

15. The filled capsules of step 14 were band-sealed using the gelatin solution.

### Dissolution Studies (USP Test-III)

Dissolution tests were carried out using Example 27, 28, 29 and 30. The dissolution was carried out in a USP type II apparatus (with alternative sinkers) in 900 mL of pH 8.0 borate buffer (0.5% cetrimide and 50 mg/L pancreatin) at a temperature of 37° C.±0.5° C. The samples were taken at predefined time points and analyzed by high performance liquid chromatography (HPLC)/UV. The results of the dissolution tests are shown in Table 1.

TABLE 1
Percent drug release of Example 27, 28, 29 and 30
Drug Release (DR) profiles of different formulation examples
were compared to establish modified-release composition of
isotretinoin suitable for once-daily administration.

| | |
|---|---|
| Dissolution Media | Borate Buffer (pH 8.0) containing 0.5% cetrimide |
| (USP Test-3) | and 50 mg/L of pancreatin |
| Apparatus/ | USP Type II (with alternative sinkers)/75/900 mL |
| RPM/Vol | |

| | Percent Drug Release | | | |
|---|---|---|---|---|
| Time (Hrs) | Example 27 | Example 28 | Example 29 | Example 30 |
| 1 | 40 | 38 | 35 | 39 |
| 2 | 50 | 55 | 42 | 43 |
| 4 | 54 | 59 | 64 | 48 |
| 6 | 58 | 64 | 69 | 54 |
| 8 | 62 | 69 | 74 | 60 |
| 10 | 65 | 74 | 79 | 66 |
| 12 | 69 | 79 | 83 | 72 |
| 15 | 74 | 86 | 88 | 81 |
| 18 | 79 | 92 | 92 | 89 |
| 24 | 87 | 100 | 97 | 99 |

### Stability Data

Table 2 provides the impurity profile of Examples 27-30 when stored at a temperature of 40° C. and 75% relative humidity for the period of one month. Impurities were determined using HPLC system.

TABLE 2
Stability data of isotretinoin when stored at a temperature of 40° C.
and 75% relative humidity for the period of one month.

| | Example | | | | | |
|---|---|---|---|---|---|---|
| | Example 27 | | Example 28 | | Example 30 | |
| Related substances | Initial | 1 month | Initial | 1 month | Initial | 1 month |
| 5,6-Epoxy-13-cis | 0.19 | 0.32 | 0.24 | 0.18 | 0.16 | 0.19 |
| retinoic acid | | | | | | |
| Total Impurities | 0.40 | 0.87 | 0.48 | 0.56 | 0.28 | 0.40 |

We claim:

1. A modified-release, oral pharmaceutical composition comprising isotretinoin or a pharmaceutically acceptable salt, ester or a derivative thereof; and a release modifying agent, wherein the composition is suitable for once daily administration.

2. A modified-release, oral pharmaceutical composition comprising isotretinoin, wherein the composition comprises an immediate-release component; and a modified-release component comprising a release modifying agent, and wherein the composition is suitable for once daily administration.

3. The modified-release, oral pharmaceutical composition according to claim 2, wherein the ratio of the isotretinoin in the immediate-release component and modified-release component is about 10:90 to about 70:30.

4. The modified-release, oral pharmaceutical composition according to claim 2, wherein the immediate-release component and modified-release component are in the form of a solution, suspension, emulsion, nanoemulsion, solid particles adsorbed onto a carrier substrate, spray-dried particle dispersion, solid dispersion, powder, granules, pellets, minitablets, microcapsules, spheroids, capsules, or tablets.

5. The modified-release, oral pharmaceutical composition according to claim 2, wherein the said immediate-release component and modified-release component are in the form of a matrix tablet, reservoir type tablet, inlay tablets or bilayer tablet.

6. The modified-release, oral pharmaceutical composition according to claim 2, wherein the modified-release component or immediate release component comprises a solid dispersion of isotretinoin.

7. The modified-release, oral pharmaceutical composition according to claim 2, wherein the modified-release component is gastro-retentive.

8. The modified-release, oral pharmaceutical composition according to claim 1, wherein the oral pharmaceutical composition is in the form of solid composition or liquid composition.

9. The modified-release, oral pharmaceutical composition according to claim 1, wherein the solid composition is in the form of a tablet or a capsule.

10. The modified-release, oral pharmaceutical composition according to claim 1, wherein the composition is in the form of a capsule, wherein the capsule comprising an immediate-release tablet, and a modified-release tablet.

11. The modified-release, oral pharmaceutical composition of claim 10, further comprising a delayed-release tablet.

12. A modified-release, oral pharmaceutical composition comprising isotretinoin or a pharmaceutically acceptable salt, ester or a derivative thereof; and a release modifying polymer, wherein the ratio of isotretinoin to release modifying polymer in the composition is from about 1:1 to about 1:40, and wherein the composition is suitable for once daily administration.
